(19) United States
(12) Patent Application Publication
(10) Pub. No.: US 2025/0009690 A1
(43) Pub. Date: Jan. 9, 2025

(54) USE OF LACTIC ACID IN POSTMENOPAUSAL WOMEN

(71) Applicant: DR. AUGUST WOLFF GMBH & CO. KG ARZNEIMITTEL, Bielefeld (DE)

(72) Inventors: Manuel HÄUSER, Bielefeld (DE); Clarissa MASUR, Bielefeld (DE); Bernhard NEUHAUS, Bielefeld (DE)

(21) Appl. No.: 18/576,983

(22) PCT Filed: Jul. 6, 2022
(86) PCT No.: PCT/EP2022/068778

§ 371 Date: Feb. 9, 2024

(30) Foreign Application Priority Data
Jul. 7, 2021 (DE) 10 2021 117 562.3

(51) Int. Cl. A61K 31/19; A61K 9/00; A61K 9/02; A61K 9/107; A61K 47/06; A61K 47/10; A61K 47/12; A61K 47/14; A61K 47/34; A61K 47/36; A61P 15/02; A61P 15/12

(52) U.S. Cl. CPC A61K 31/19; A61K 9/0034; A61K 9/02; A61K 9/107; A61K 47/06; A61K 47/10; A61K 47/12; A61K 47/14; A61K 47/34; A61K 47/36; A61P 15/02; A61P 15/12

(57) ABSTRACT

The present invention relates to the use of lactic acid and/or lactic acid salts for the treatment and/or prevention of estrogen deficiency-related vaginal symptoms, urogenital menopausal syndrome, postmenopausal atrophic vaginitis, senile vaginitis, vulvovaginal atrophy and/or vaginal dryness in postmenopausal women.

The present invention relates to the use of lactic acid and/or lactic acid salts for the treatment and/or prevention of estrogen deficiency-related vaginal symptoms, urogenital menopausal syndrome, postmenopausal atrophic vaginitis, senile vaginitis, vulvovaginal atrophy and/or vaginal dryness in postmenopausal women.

The physiological pH of a healthy vagina is approximately 4. It is adjusted by the presence of lactic acid, which is produced in the vagina from glycogen by lactobacilli. In this slightly acidic environment, the growth of other germs is made more difficult and, among other things, the vagina is protected from bacterial infections. If this protection is lost or significantly weakened, vaginal infections occur more easily. One approach in the treatment and prevention of various bacterial diseases such as bacterial vaginosis (Gardnerella vaginitis, vaginal inflammation) is therefore the application of lactic acid, which is intended to adjust the acidic pH value and support the vaginal flora accordingly. The lactic acid is usually applied or introduced into the internal genital area in the form of creams or suppositories. Lactic acid is also often contained in products for the non-hormonal treatment of vaginal dryness in order to adapt the pH value of the products in question to the requirements of the vaginal environment.

As women age, estrogen levels decrease, often leading to shrinkage of the woman's genital area and, in particular, thinning of the vaginal epithelial tissue and symptoms such as vaginal dryness and pain. This phenomenon is called vaginal atrophy and occurs particularly in women during and after menopause. Typically, vaginal atrophy is treated by systemic or local hormone administration (estrogen therapy), with local application showing a better side effect profile due to the lower dosage. Alternatives to estrogen therapy are non-hormonal treatments such as the use of lubricants and moisturizing gels or laser therapy.

Lee, Y. K., et al. (2011) “Vaginal PH-balanced gel for the control of atrophic vaginitis among breast cancer survivors: a randomised controlled trial.” Obstet Gynecol 117(4): 922-927 and Kim, Y H, et al. (2017). “Effect of a pH-Balanced Vaginal Gel on Dyspareunia and Sexual Function in Breast Cancer Survivors Who Were Premenopausal at Diagnosis: A Randomized Controlled Trial.” Obstet Gynecol 129(5): 870-876 have also found that application of a gel containing 1% lactic acid alleviates vaginal symptoms such as vaginal dryness and dyspareunia in artificially postmenopausal women (breast cancer patients). The reason given is that the vaginal pH value is adjusted to around 4 by lactic acid. Regardless of this, there are various market products (such as Vagisan FeuchtCreme Cremolum from Dr. Wolff) for the treatment of vaginal dryness, which contain lactic acid to adjust the vaginal pH value and thus contribute to maintaining a healthy vaginal environment for women of all ages. However, there is still a need to provide effective and low-side effect treatment options for vaginal atrophy explicitly in naturally postmenopausal women.

The object underlying the present invention was therefore to provide effective hormone-free prevention and therapy of vaginal atrophy in postmenopausal women.

This object was surprisingly achieved according to the present invention as defined in the patent claims. In particular, it has surprisingly been found that by using lactic acid and/or lactic acid salts in the claimed dosage not only vaginal (vulvovaginal) atrophy (VVA), but also the urogenital menopause syndrome (Genitourinary Syndrome of Menopause: GSM), postmenopausal atrophic vaginitis, senile vaginitis, vaginal dryness and other vaginal symptoms caused by estrogen deficiency can be effectively treated and prevented. Unexpectedly, there was a significant improvement in cellular differentiation (related to parabasal cells, intermediate cells and superficial cells of the vaginal tissue), expressed by the VMI as described below. A clear influence on cellular differentiation has so far only been observed with hormone-containing preparations. Lee et al. (see above) found only slightly changed cell differentiation with their hormone-free gel. Also unexpected was the improvement in the quality of life of the postmenopausal women treated according to the invention, which surprisingly was even found to be independent of symptoms (determined by various validated tests such as the DIVA test/questionnaire described below).

The compositions used according to the invention contain lactic acid and/or lactic acid salts (lactates) as active ingredients. Suitable lactic acid salts include the alkali metal lactates (in particular lithium, sodium and/or potassium lactate) or the alkaline earth metal lactates (in particular calcium, magnesium and/or barium lactate) as well as ammonium lactate and lactates with various organic cations. The lactic acid and lactic acid salts are preferably monolactic acid or monolactic acid salts, because in this way a preferred immediate release of the active ingredient (i.e., not a delayed release) is achieved. The dosage of lactic acid is higher than known in the prior art. The higher dosage apparently also leads to the improvement in cellular differentiation in the vaginal epithelial tissue, which was surprisingly found according to the invention. In contrast to the prior art, the effect of lactic acid is completely unexpected not only in the adjustment or maintenance of the acidic pH value to support the vaginal flora, but instead even shows a cellular influence.

According to the invention, the lactic acid and/or the lactic acid salts are applied in an amount of at least 120 mg per dosage of the composition. “Per dosage” means, inventively, per quantity (weight) of the composition applied at the time of use (e.g., per dose or, if several doses are administered simultaneously, per total quantity of doses applied or per total amount of emulsion/cream applied). According to the invention, the amount of lactic acid and/or lactic acid salts applied always refers to “lactic acid equivalents (anhydrous),” that is, it is based on the equivalent amount of anhydrous lactic acid and, if necessary, converted to this. Application in an amount of 120 to 5000 mg, particularly preferably 140 to 1000 mg, more preferably 160 to 500 mg, very particularly preferably 180 to 200 mg is preferred. A typical and particularly effective application amount is approximately 190 mg per dosage. At an amount below 120 mg per dosage there was an insufficient influence on cellular differentiation. If the amounts were too high, the dosage units were not sufficiently stable and the active ingredient distribution was not sufficiently homogeneous, resulting in undesirable deviations in the dosage.

To enable this dosage, the compositions preferably contain at least 6.0% by weight of lactic acid and/or lactic acid salts, based on the total weight of the composition. Preferred is a range from 6 to 15% by weight, particularly preferably from 6 to 12% by weight, most preferably from 6 to 10% by weight of lactic acid, based on the total weight of the composition.

The vaginal use according to the invention includes both the topical application of the composition to the internal and/or external genital area in the form, for example, of semi-solid formulations (including creams, water-in-oil (W/O) or oil-in-water (O/W) emulsions, gels, etc.) as well as the intravaginal introduction of the composition, for example, in the form of suppositories or ovules. According to the invention, the use takes place by means of the methods known in the prior art. It is particularly preferred to use it as a cream (particularly as an O/W emulsion) or as a (vaginal) suppository. Both forms allow use in the internal and external genital area, whereby the suppositories are not actively applied in the external genital area, but rather partially leak out after melting/dissolving in the vagina and thus reach the external genital area. Emulsions, on the other hand, are applied directly to the internal and/or external genital area. The compositions particularly preferably contain no hormones. In other words, the use according to the invention is hormone-free overall.

Emulsions preferably used according to the invention include an aqueous phase, an oil phase and an emulsifier system. Oil-in-water emulsions (O/W) are particularly preferred. The composition of the oil phase is not critical and is formed from commonly used oil components. The emulsifier system comprises (or consists of): at least one nonionic polyoxyethylene ether of one or more fatty alcohols, at least one fatty acid and at least one glycerol fatty acid ester. Alternatively, the emulsifier system comprises (or consists of): at least one nonionic polyoxyethylene ether of one or more fatty alcohols, at least one fatty acid and at least one fatty alcohol.

Preferred emulsifier systems comprise (or consist of): a) at least one nonionic polyoxyethylene stearyl ether, at least one fatty acid with 10 to 20 C atoms, and glycerol monostearate: or b) at least one nonionic polyoxyethylene stearyl ether, at least one fatty acid with 10 to 20 C atoms, and at least one fatty alcohol with 10 to 20 C atoms.

Suppositories preferably used according to the invention (also generally called suppositories, vaginal suppositories or ovules) can be both water-soluble and fat-containing preparations. A base commonly used for fatty suppositories is hard fat with a melting range close to human body temperature. For example, a semi-synthetic mixture of mono-, di- and triglycerides is suitable according to the invention. Suppositories containing fat melt at body temperature and release the active ingredient from the melt. Unlike fatty suppositories, water-soluble suppositories are not intended to melt at body temperature. Rather, they dissolve, for example, in the water present in the vagina and release the active ingredient there. Mixtures of different polyethylene glycols (Macrogols such as Macrogol 1500 and Macrogol 6000) are suitable as carrier materials. Both types of suppositories and the preferred components used therein are fundamentally known to those skilled in the art.

The pH of the compositions is preferably 3 to 6, particularly preferably 4-5 and most preferably about 4. It is preferably adjusted using one or more pH modifiers. Suitable pH modifiers can be acids, bases and/or buffer systems to stabilize or influence the pH of the composition. Typical pH modifiers according to the present invention are adipic, citric, malic, succinic, tartaric, ascorbic, phosphoric, lactic and fumaric acids and the corresponding salts, as well as sodium alginate, polyacrylic acid, sodium hydroxide, sodium carbonate and sodium bicarbonate. In the context of pH modifiers, the term salt refers to alkali metal salts or alkaline earth metal salts unless otherwise specified.

In a further embodiment, the composition contains at least one additive. Additives that are commonly used in vaginal products (and in particular in creams and suppositories) are preferred. The at least one additive can be present in a proportion of 0.01% by weight to 12.0% by weight, more preferably from 0.25% by weight to 10.0% by weight, in particular from 1.0 to 7.0% by weight are present.

In addition, the at least one additive can be selected from the group consisting of preservatives, stabilizers, fragrances, antioxidants, rheology modifiers, thickeners, cosmetic care products, dyes, brightening agents, solvents and combinations thereof.

Preservatives are substances used for preservation by killing and/or inhibiting the growth of microorganisms that break down the composition. Preferably, the preservatives may be selected from the group consisting of benzyl alcohol, benzoic acid, benzoic acid derivatives, sorbic acid, sorbic acid derivatives, salicylic acid, salicylic acid derivatives, phenoxyethanol, parabens and combinations thereof. In a preferred embodiment, benzyl alcohol is used as a preservative.

Stabilizers can protect light-sensitive components from radiation and are preferably UV absorbers such as benzophenone derivatives.

The addition of fragrances can provide a pleasant smell to the composition. Examples are the perfumes known to the person skilled in the art.

An antioxidant or antioxidation product is a chemical compound that slows or completely prevents oxidation of other components in the composition used according to the invention. Examples of suitable antioxidants include citric acid, ascorbic acid and butylhydroxyanisole.

Rheology modifiers and thickeners can help to improve the application properties of the composition according to the invention. The addition of table salt (sodium chloride) as a rheology modifier and thickener can be considered. By adding table salt, the flowability of the composition according to the invention can be influenced within certain limits and adjusted to the necessary level. Polysaccharide derivatives or polyacrylates can also be used as thickeners.

A solvent or solvent mixture that is well known by those skilled in this field can be used as the solvent. The preferred solvent is water.

As described above, the use according to the invention relates to the medical treatment and/or prevention of vaginal symptoms caused by estrogen deficiency, the urogenital menopause syndrome (Genitourinary Syndrome of Menopause: GSM), postmenopausal atrophic vaginitis, senile vaginitis, vulvovaginal atrophy (VVA) and/or vaginal dryness. Patients with these indications are women after menopause (postmenopausal women). In principle, the term “postmenopausal” includes both women with natural menopause (ICD-10 classification N95.2) and women with artificial menopause (ICD-10 classification N95.3). An artificial menopause is caused artificially, for example by ovariectomy or treatment with antiestrogens or drug therapy that influences the woman's hormonal balance. A non-limiting example is the treatment of breast cancer patients with tamoxifen. Naturally menopausal women (i.e., naturally postmenopausal women or non-artificially postmenopausal women) are the preferred patient group according to the present invention.

The use of higher doses of lactic acid according to the invention surprisingly leads to an improvement in the subjective symptoms of VVA/GSM. The improvement in subjective symptoms can be recorded, for example, by a subjective symptom sum score, which consists of the individual symptoms of dryness, itching, burning and pain, regardless of sexual intercourse, each rated on a scale of 0-4 (0=not present: 4=extreme). A lower subjective symptom sum score therefore means an improvement in VVA/GSM.

Furthermore, the use of higher doses of lactic acid according to the invention led to an improvement in the objective signs of VVA/GSM, which can be determined by a doctor. The vaginal health index (VHI), for example, is used to quantify/evaluate the objective signs and consists of the joint evaluation of the individual parameters general elasticity, type of secretion and consistency, pH value, epithelial mucosa and moisture. Each parameter can have a value of 1-5, where 5 corresponds to excellent properties and a value of 1 describes the highest degree. The VHI is presented as the sum score of the individual parameters, so a higher value means an improvement.

In particular, the use according to the invention of higher doses of lactic acid and/or lactic acid salts surprisingly leads to an improvement in cellular differentiation (based on parabasal cells, intermediate cells and superficial cells of the vaginal tissue), expressed by the vaginal maturation index (VMI). According to the invention, the VMI is defined as follows:

VMI=1.0x % (superficial cells)+0.5x % (intermediate cells)+0.0x % (parabasal cells)

The parabasal cells are not taken into account in this formula. The difference in the vaginal maturation index (VMI) between before and after the treatment according to the invention, i.e., the improvement in the VMI, is at least 10, preferably 20 to 40, particularly preferably 25 to 30. The women to be treated particularly preferably have a vaginal maturation index before treatment (VMI) of less than 35. While, as mentioned, hormonal treatments have an influence on the cellular differentiation of the vaginal epithelium, this has not yet been proven for non-hormonal treatments in the state of the art for postmenopausal women based on the VMI.

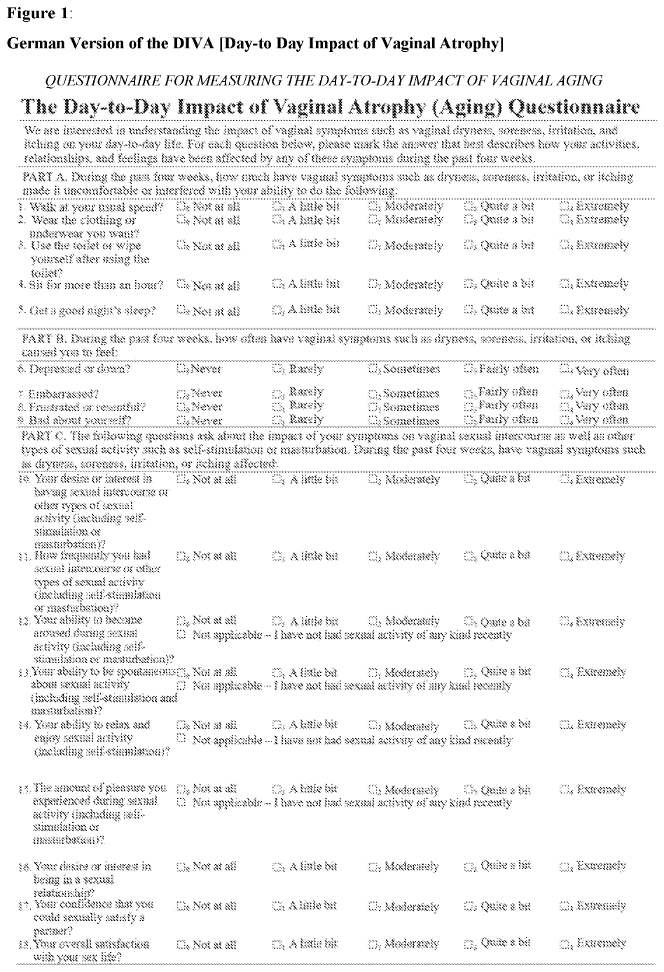

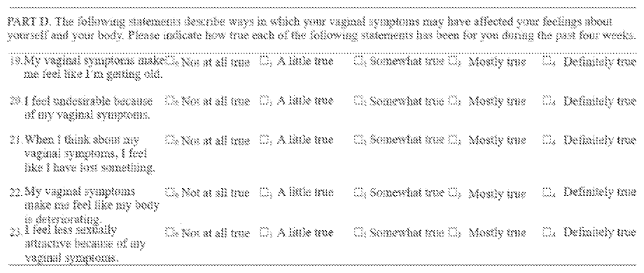

The improvement in the quality of life of the treated postmenopausal women associated with the invention was equally unexpected. Surprisingly, this was even symptom-independent, as determined by the DIVA test/questionnaire (DIVA=Day-to-day Impact of Vaginal Aging). The DIVA questionnaire (FIG. 1) addresses the impact of vaginal symptoms (vaginal dryness, irritation, itching, soreness) on patients' daily lives in terms of their daily activities, relationships and feelings. According to the invention, an improvement in quality of life is achieved if the resulting value from the DIVA questionnaire shows a statistically significant reduction in the DIVA total score. The reduction in the total DIVA score is at least 0.5, preferably 0.5 to 16, particularly preferably 1 to 10 and most preferably 2, 3, 4, 5 or 6 units. In addition to the DIVA questionnaire, other validated tests for assessing quality of life are also suitable according to the invention. Equally surprisingly, the use of higher doses of lactic acid according to the invention in postmenopausal women with VVA/GSM only leads to moderate to mild adverse events and is generally well to very well tolerated.

### EXAMPLES

The following Formulation Examples 1-3 are suitable for use in accordance with the present invention.

### Example 1: O/W Emulsion

| Ingredient | Quantity [g] |
|---|---|
| Beeswax | 0.20 |
| Steareth-2 | 3.00 |
| Steareth-21 | 2.00 |
| Decyl oleate | 6.50 |
| Glycerol monostearate 40-50 | 4.00 |
| Stearic-palmitic acid | 3.00 |
| Benzyl alcohol | 1.00 |
| Lactic acid 90% | 8.90 |
| Water, purified | 56.40 |
| NaOH 30% | q.s. |
| Water, purified | 15.00 |
| | 100.00 |

The formulation has a pH of 4.0.

### Example 2: O/W Emulsion

| Ingredient | Quantity [g] |
|---|---|
| Steareth-2 | 3.00 |
| Steareth-21 | 2.00 |
| PPG-15 Stearyl Ether | 4 |
| Isohexadecane | 5.00 |
| Cetylstearyl alcohol | 1.00 |
| Stearic-palmitic acid | 1.50 |
| Dimethicone 350 | 1.00 |
| Benzyl alcohol | 1.00 |
| Lactic acid 90% | 8.90 |
| Water, purified | 52.90 |
| Propylene glycol | 4.00 |
| NaOH 30% | q.s. |
| Water, purified | 15 |
| Xanthan gum | 0.70 |
| | 100.00 |

The formulation has a pH of 4.0.

### Example 3: Suppositories

| Ingredient | Amount [mg] |
|---|---|
| Macrogol 1500 | 2050.00 |
| Macrogol 6000 | 683.00 |
| Lactic acid 90% | 167.00 |
| Sodium lactate solution 50% | 100.00 |
| | 3000.00 |

### Example 4: Determination and Significance of the VMI Index

The following study was conducted.

The use of a preparation according to the invention with a high lactic acid content (according to Example 3) was examined in 42 female patients suffering from the symptoms of vulvovaginal atrophy. All patients were postmenopausal at the start of the study and had a subjective symptom sum score—consisting of dryness, itching, burning and pain independent of sexual intercourse, each rated from 0-4—of at least 3. In addition, the patients had a VMI≤35.

The intravaginal application of the lactic acid according to the invention was performed daily for the first week (preferably in the evening before going to bed). In the following five weeks, the application was carried out twice a week.

The patients were examined gynecologically at the start of the study and at least after six weeks. In the process. at least the subjective symptom sum score for vulvovaginal atrophy was recorded, an objective assessment within the framework of the VHI and VMI was recorded, and the DIVA questionnaire was completed.

The vaginal maturation index (VMI) describes the degree of differentiation of the epithelial cells of the vaginal epithelium (composition of parabasal cells, intermediate cells and superficial cells). For this purpose, the ratio of superficial cells to intermediate cells and parabasal cells is determined microscopically. A description of the VMI can be found in Willhite, LA and MB O'Connell (2001). “Urogenital Atrophy: Prevention and Treatment.” Pharmacotherapy 21 (4): 464-480.

### Calculation:

$$VMI = 1. \times \% superficial cells + 0.5 \times \% intermediate cells + 0. \times \% parabasal cells$$

The change in VMI between day 1 and day 43 was statistically significant. Thus, the use according to the invention of the preparation containing lactic acid has a clear influence on the differentiation of the cells of the vaginal epithelium.

| Mean VMI (±statistical | | Mean difference | p-value (Day 43 |
|---|---|---|---|
| deviation [SA]) | | (day 43 vs. day 1) | vs. Day 1) |
| Day 1 | 8.3 (±12.2) | 26.6 | <0.0001 |
| Day 43 | 34.9 (±19.5) | | |

### Example 5: Assessment of Improvement in Quality of Life with DIVA

The DIVA, in German “Questionnaire on the Everyday Effects of Vaginal Atrophy (of vaginal aging)”, is composed of four scales (see FIG. 1).

Scale 1 refers to the impact of the symptoms on activities of daily living, Scale 2 on emotional well-being, Scale 3 examines the influence of problems on sexual functioning, although in this case there is a short version for all women (including non-sexually active women) and a longer version for sexually active women, and Scale 4 reflects the effects on self-concept and body image.

The questionnaire consists of 23 items, with 4 of these items only to be answered by recently sexually active women. Each item can be rated on a scale of 0 to 4.

The overall score of a scale is determined by the mean of the respective items (sum of the items in a scale/number of items in the scale). So the overall score for each scale is between 0 and 4. The DIVA total score is the sum of the total scores of the individual scales. The higher the score, the greater the perceived impact of vaginal symptoms. The recall period covers the last four weeks.

In addition to English, the questionnaire is currently available in Spanish and Italian.

The German version of the DIVA (see FIG. 1) was created by a translation agency and linguistically validated using interviews with the target population, i.e., postmenopausal women with vaginal complaints.

| | Mean overall DIVA score | Standard deviation |
|---|---|---|
| Day 1 | 5.45 | 2.80 |
| Day 43 | 2.57 | 1.97 |

The reduction in the total score (=2.9) from day 1 to day 43 is statistically significant. In addition, the reduction in the individual domain scores is also statistically significant. An improvement in the quality of life of the patients was thus shown through the use of lactic acid according to the invention.

1. A method for treating or preventing estrogen deficiency-related vaginal symptoms, urogenital menopausal syndrome, postmenopausal atrophic vaginitis, senile vaginitis, vulvovaginal atrophy and/or vaginal dryness in a subject, comprising administrating a composition containing lactic acid and/or one or more lactic acid salts to the subject, wherein the lactic acid and/or the one or more lactic acid salts are applied in an amount of at least 120 mg per dosage of the composition.

2. The method according to claim 1, wherein the subject to be treated is a naturally (non-artificial) postmenopausal woman.

3. The method according to claim 1,
wherein the administration of the composition results in a difference in a vaginal maturation index (VMI) in the subject, wherein the difference in the vaginal maturation index (VMI), between before and after treatment is at least 10.

4. The method according to claim 1, wherein the administration of the composition results in an improvement in the quality of life, determined with a Day-to-day Impact of Vaginal Aging (DIVA) questionnaire, wherein the improvement in the quality of life has a statistically significant reduction in the total DIVA score by at least 0.5 units.

5. The method according to claim 1, wherein the composition is selected from an emulsion comprising an aqueous phase, an oil phase and an emulsifier system, and a suppository.

6. The method according to claim 5, wherein the composition is an emulsion having an emulsifier system, wherein the emulsifier system comprises:
at least one nonionic polyoxyethylene ether of one or more fatty alcohols,
at least one fatty acid, and
at least one glycerol fatty acid ester.

7. The method according to claim 5, wherein the composition is an emulsion having an emulsifier system, wherein the emulsifier system comprises:
at least one nonionic polyoxyethylene stearyl ether,
at least one fatty acid with 10 to 20 carbon atoms, and
glycerol monostearate.

8. The method according to claim 1, wherein the composition is a water-soluble suppository which has a mixture of at least two different polyethylene glycols as a carrier material.

9. The method according to claim 1, wherein the composition does not contain any hormones.

10. The method according to claim 1, wherein the lactic acid is monolactic acid.

11. The method according to claim 1, wherein the composition is applied topically to the internal and/or external genital area and/or introduced into the vagina.

12. The method according to claim 1, wherein the composition is not a gel.

13. The method according to claim 5, wherein the composition is an emulsion having an emulsifier system, wherein the emulsifier system consists of:
at least one nonionic polyoxyethylene ether of one or more fatty alcohols,
at least one fatty acid, and
at least one glycerol fatty acid ester.

14. The method according to claim 5, wherein the composition is an emulsion having an emulsifier system, wherein the emulsifier system comprises:
at least one nonionic polyoxyethylene ether of one or more fatty alcohols,
at least one fatty acid, and
at least one fatty alcohol.

15. The method according to claim 5, wherein the composition is an emulsion having an emulsifier system, wherein the emulsifier system comprises:
at least one nonionic polyoxyethylene ether of one or more fatty alcohols,
at least one fatty acid, and
at least one fatty alcohol.

16. The method according to claim 5, wherein the composition is an emulsion having an emulsifier system, wherein the emulsifier system consists of:
at least one nonionic polyoxyethylene stearyl ether,
at least one fatty acid with 10 to 20 carbon atoms, and
glycerol monostearate.

17. The method according to claim 5, wherein the composition is an emulsion having an emulsifier system, wherein the emulsifier system comprises:
at least one nonionic polyoxyethylene stearyl ether,
at least one fatty acid with 10 to 20 carbon atoms, and
at least one fatty alcohol with 10 to 20 carbon atoms.

18. The method according to claim 5, wherein the composition is an emulsion having an emulsifier system, wherein the emulsifier system consists of:
at least one nonionic polyoxyethylene stearyl ether,
at least one fatty acid with 10 to 20 carbon atoms, and
at least one fatty alcohol with 10 to 20 carbon atoms.

19. The method according to claim 1, wherein the method is entirely hormone-free.

20. The method according to claim 1, wherein the lactic acid is not oligolactic acid or polylactic acid.
